(19) United States
(12) Patent Application Publication
(10) Pub. No.: US 2026/0053412 A1
(43) Pub. Date: Feb. 26, 2026

(54) PELVIC FLOOR STIFFNESS MEASURING DEVICE APPLYING PHYSICAL METHOD

(71) Applicant: HEEJU HAN, Cheongju-si (KR)

(72) Inventors: HEEJU HAN, Cheongju-si (KR); JAEHOON HAN, Sejong-si (KR); MINSEO KIM, Tongyeong-si (KR)

(21) Appl. No.: 18/948,293

(22) Filed: Nov. 14, 2024

Related U.S. Application Data
(63) Continuation of application No. PCT/KR2024/014906, filed on Sep. 30, 2024.

(30) Foreign Application Priority Data
Aug. 20, 2024 (KR) 10-2024-0111376

(51) Int. Cl. A61B 5/22; A61B 5/00

(52) U.S. Cl. CPC A61B 5/227; A61B 5/6879; A61B 2562/0247

(57) ABSTRACT

Provided is a pelvic floor stiffness measuring device including: based on a longitudinal direction, a pair of pressure members including insertion parts formed at each end thereof, grip parts formed at each other end of the pair of pressure members, and a hinge part that does not intersect between the insertion part and the grip part, the pair of insertion parts being open/closed by opening/closing the grip parts around the hinge part, in which the pair of insertion parts has each one end forming a pelvic floor pressure area based on the longitudinal direction and each other end connected to the hinge part, and has a downward convex curved surface based on a widthwise axis of symmetry through which the pair of pressure members is open/closed, and is connected by an intermediate area in which a point where the pair of insertion parts intersect is formed at least in part.

### TECHNICAL FIELD

The present disclosure relates to a portable pelvic floor stiffness measuring device applying a physical method, which is designed to be inserted into a pelvic cavity for application.

### BACKGROUND ART

“Pelvic floor” refers to pelvic floor muscles of a pelvic floor, and is composed of muscle fibers of pubococcygeus muscle, levator ani, coccygeus muscle, and related connective tissue that span a lower portion of a pelvis as pelvic floor muscles or pelvic floor part. The pelvic floor is a muscle that separates an upper pelvic cavity from a lower perineum (including genitals and anus), and supports organs such as uterus, bladder, and rectum while surrounding the genitals like a hammock (see (b) of FIG. 5).

As described above, one of the major roles of the pelvic floor muscles is ‘support and hold.’ The pelvic floor muscles are connected to muscles located in an abdomen to hold the organs so that they do not sag downward. In addition, the pelvic floor muscles are attached to an end portion of a tailbone, helping to straighten a waist. In other words, a central portion of a body is stabilized by the support and holding. In addition, the pelvic floor muscles surround urethra, vagina, and anus opening. The pelvic floor muscles are used to tighten and relax the anus and urethra to suppress or expel urine and feces, and contraction of the pelvic floor muscles also affects orgasm during sexual intercourse.

The pelvic floor muscles are naturally subjected to an aging process as people get older. The pelvic floor muscles do not need to exert force due to lifestyle habits such as sitting at a desk for a long period of time or riding in a car, which causes the pelvic floor muscles to weaken. In particular, in the case of women, during pregnancy, weights of a baby and an amniotic fluid is concentrated as a load and stress is accumulated on the pelvic floor muscles that support the weight, during childbirth, the pelvic floor muscles are damaged due to incisions, etc. Women experience menopause without properly recovering the pelvic floor muscles that have been weakened due to the pregnancy and childbirth, and are subjected to serious problems as their pelvic floor muscles have significantly reduced functions due to the effect of hormones.

Muscle tone of the pelvic floor is a state of muscles at rest. Clinically, the muscle tone is commonly used as a term meaning a degree of stiffness of a tissue, i.e., tissue stiffness, as “a movement distance of a tissue in response to a passive external force.” Biomechanically, the muscle tone is defined as the resistance of the tissue when the external force is applied, and may be measured with stiffness.

The tone includes contractile factors such as muscles and non-contractile factors such as fascia, tendons, and connective tissue. A method and criteria for measuring tone of a pelvic floor are not standardized (Rachel Worman, 2023), but contractile elements are mainly measured using electromyography, and the tissue itself including non-contractile elements is measured using pressure-based measurement methods such as perineometer, dynamometer, manometry, etc.

The physical measurement methods are combined with expert opinions on tissue stiffness in response to pressure using measurement methods such as 2D, 3D Ultrasound (Shearwave elastography), MRI, and finger palpation to make a diagnosis for the tone. However, the pressure-based measurement is to primarily measure the strength of contractions in pelvic floor tissues, such as sphincters, and is a physically different characteristic from the stiffness of the tissue. There is currently no physical measuring device developed that measures the levator ani muscle, which acts as main muscles supporting pelvic floor tissues at a vertical angle, rather than just a sphincter muscle in a round bowl-shaped pelvic floor muscle.

Related Art 1 (EP 2689724 A1) relates to a pelvic floor muscle strength measuring device, and discloses a speculum including: a grip area 31 and 32; pivotally coupled parts 11 and 12; and front areas 41 and 42 introduced into vagina, in which the grip area 31 and 32 of the speculum includes a movement sensor 2 coupled with a spring 21 having a stiffness constant K, and is connected to a module 50 for reading movement. However, Related Art 1 has limitations in that the structure of the speculum is specific to the pelvic floor sphincter, the sphincter is a part of a structure corresponding to a second layer of three layers of the pelvic floor muscles, and measuring the generation of a tightening force (muscle force) is a completely different characteristic from non-contractile stiffness of a tissue.

Related Art 2 (KR 0904751 B1) is to extract parameters such as maximum contraction pressure and maximum pressure duration from a contraction pressure of pelvic floor muscles, and relates to a pressure measuring device including a balloon sensor for vaginal insertion and a pressure sensor that may quantify and express the pressure. However, Related Art 2 is to measure the contraction pressure of the pelvic floor muscles, and like Related Art 1, is to measure the generation of the tightening force (muscle force) throughout the vagina, so it is practically impossible to measure the non-contractile stiffness of the pelvic floor tissue with the corresponding device.

Related Art 3 (KR 2024-0035222A) discloses a pressure detection sensor that is inserted into vagina to measure pressure inside a body cavity, and is a technology related to a treatment device for urinary and fecal incontinence that includes an electrode part that directly contacts pelvic floor muscles to measure muscle strength and apply an electrical stimulation signal. However, Related Art 3 relates to an electromyography technology used for measuring contractile elements (muscles), and has a problem in that it is not suitable for measuring a pelvic floor tissue itself, including non-contractile elements.

Accordingly, there is a need for the development of a portable measuring device capable of screening by measuring a pressurizable pressure value and a movement distance of a tissue from the time of contact to an end point of pressurization when a certain pressure is applied to the tissue in a pelvic cavity according to the definition of stiffness.

### DISCLOSURE

### Technical Problem

The present disclosure provides a pelvic floor stiffness measuring device capable of providing objective data and improving portability and accessibility by applying a simple, scissors-shaped diagnostic and measuring tool that reaches levator ani, which corresponds to a main muscle layer in a pelvic cavity, and includes a pressurizable pressure sensor and a movement sensor that can measure a non-contractile displacement of a tissue.

Specifically, the present disclosure is to calculate tissue stiffness by measuring a movement distance by a non-stretchable tissue when a pair of insertion parts spaced apart in a width direction press both wall surfaces of a pelvic floor tissue and pressure at this time as a user opens and closes (progresses) a grip part of a scissors-shaped measuring device in the width direction to open and close the pair of insertion parts inserted into vagina or anus around a hinge part while being in contact with a body tissue in a pelvic cavity.

In addition, the present disclosure is to improve the problem of pressure being distributed during an operation of the measuring device by efficiently contacting and pressurizing only (horizontal) both wall surfaces of a pelvic floor tissue targeted by a scissors-type pressure member and not contacting other internal body tissues other than the pelvic floor.

In addition, the present disclosure is to improve a problem in which pressure is dispersed when a measuring device is operated by preventing contact and pressurizing with other internal body tissues in a front reference thickness (vertical) direction of a body as levator ani of a deep structure of a pelvic floor to measure stiffness is located in lower portions in 4-5 o'clock and 7-8 o'clock directions when a front of an introitus or an anus opening is approximated as a uniform circle.

### Technical Solution

According to one implementation example of the present disclosure, a pelvic floor stiffness measuring device includes: based on a longitudinal direction, a pair of pressure members (10) that includes insertion parts (100) formed at each end of the pair of pressure members (10), grip parts (200) formed at each other end of the pair of pressure members (10), and a hinge part (300) that does not intersect between the insertion part (100) and the grip part (200), the pair of insertion parts (100) being open and closed by opening and closing the pair of grip parts (200) around the hinge part (300), in which the pair of insertion parts (100) has each one end forming a pelvic floor pressure area (110) based on the longitudinal direction and each other end connected to the hinge part (300), and has a downward convex curved surface based on a widthwise axis of symmetry through which the pair of pressure members (10) is open and closed, and is connected by an intermediate area (130) in which a point (131) where the pair of insertion parts intersect is formed at least in part.

The pair of insertion parts (100) may satisfy the following Relational Expression 1.

$$0 \leq D 2 / D 1 < 1 \qquad [ Relational Expression 1 ]$$

In Relational Expression 1, D1 and D2 may each be a separation distance (D1) between the pair of pelvic floor pressure areas (110) and a separation distance (D2) between the pair of intermediate areas (130) based on the widthwise axis of symmetry of the pair of pressure members 10 when the pair of insertion parts (100) is open and closed by the opening and closing of the pair of grip parts (200), respectively.

The pair of insertion parts (100) may further include a safety area (150) in which each end is connected to the hinge part (300), and when the pair of insertion parts (100) changes from a closed state to an open state by opening and closing the pair of grip parts (200), the pair of pelvic floor pressure areas (110) may indicate a gradient in which the separation distance (D1) increases, the pair of intermediate areas (130) may indicate a gradient in which the separation distance (D2) decreases and then increases.

The pair of insertion parts (100) may be provided such that, based on a central axis in a thickness direction of the pair of pressure members (10), each pelvic floor pressure area (110) is located under the hinge part (300) and provided in an upward convex shape.

The pair of pressure members (10) may further include a sensor unit (400) that includes a movement sensor (410) measuring a movement distance of the pair of pelvic floor pressure areas (110) and a pressure sensor 420 measuring pressurized force on the pelvic floor, when the pair of insertion parts (100) is open and closed by opening and closing the pair of grip parts (200).

The pressure sensor (420) may be installed in the pelvic floor pressure area (110) of the pair of insertion parts (100) and/or at least some area of the pair of grip parts (200).

When the pelvic floor pressure area (110) starts from a point where it contacts a pelvic floor deep structure and the insertion part (100) is expanded in a width direction, the pressure applied to the pelvic floor may be measured through the pressure sensor (420) to output pressure information, and the movement distance of the pelvic floor pressure area (110) may be measured through the movement sensor (410) to output movement distance information of the tissue, and stiffness of the pelvic floor deep structure may be calculated through the pressure information and the movement distance information.

### Advantageous Effects

According to the present disclosure, it is possible to provide the useful pelvic floor stiffness measuring tool that can evaluate the pelvic floor tone by making the volume small and accurately measuring the pressure element without the finger palpation, and obtain the information on the pelvic floor tone even in the environment without expensive equipment such as ultrasound or MRI.

According to the present disclosure, i) the pair of scissors-shaped pressure members has curved shapes that contact each other but do not intersect in the hinge part connecting the insertion part and the grip part in the middle, and ii) the pair of insertion parts is contacted and intersected in the intermediate area (based on the widthwise axis of symmetry) located between the hinge part and the insertion part, so the respective widths of the end portion and the middle portion are very narrow when the measuring device is in the non-open state. Therefore, it can be inserted into a narrow vagina or anus without any special physical damage. In addition, when the inserted pressure member (insertion pressure area) is progressed (open) in the width direction, the pressure area of the insertion part increases in the separation distance (in the width direction), and the middle portion decreases and then increases in the separation distance, so the pressure distribution problem can be improved by preventing the measuring device from contacting other internal body tissues other than the pelvic floor with which the end portion is in contact.

In addition, since each pelvic floor pressure area of the vaginal insertion part is i) located below the hinge part, and ii) has an upward convex shape, the pelvic floor pressure area of the insertion part may be easily inserted downward when inserted into the vagina or anus, and even in the environment where the separation distance between both wall surfaces of the pelvic floor narrows as it goes down to the pelvic floor in the thickness direction in the vaginal or anal passage, the contact and pressure distribution to other internal body tissues other than the pelvic floor may be prevented.

### BRIEF DESCRIPTION OF THE DRAWINGS

FIG. 1 is a schematic diagram of a pelvic floor stiffness measuring device including a pair of pressure members according to one embodiment of the present disclosure.

FIG. 2A is a front schematic diagram of the pelvic floor stiffness measuring device in a closed state, based on a longitudinal direction of a pair of pressure members according to one embodiment of the present disclosure.

FIG. 2B is a front schematic diagram illustrating open and closed states of insertion parts of the pair of pressure members.

FIG. 3 is a side schematic diagram of the pelvic floor stiffness measuring device in the closed state, based on a thickness direction of the pair of pressure members according to one embodiment of the present disclosure.

FIG. 4 is a photograph illustrating the pelvic floor stiffness measuring device according to one embodiment of the present disclosure.

FIGS. 5 and 6 are reference diagrams for describing an operating state of the pelvic floor stiffness measuring device according to the present disclosure.

### BEST MODE

Advantages and features of the present disclosure and methods to achieve them will be elucidated from embodiments described below in detail with reference to the accompanying drawings. However, the present disclosure is not limited to embodiments disclosed below, and may be implemented in various different forms, these embodiments will be provided only in order to make the present disclosure complete and allow one of ordinary skill in the art to which the present disclosure pertains to completely recognize the scope of the present disclosure, and the present disclosure will be defined by the scope of the claims.

Unless defined otherwise, all terms (including technical and scientific terms) used in the present specification have the same meaning as meanings commonly understood by those skilled in the art to which the present invention pertains.

Throughout the present specification, unless described to the contrary, “including” any component will be understood to imply the inclusion of other elements rather than the exclusion of other elements. In addition, a singular form includes a plural form unless specially described in the text.

In the present specification, when a portion such as a layer, a film, a region, a plate, a structure, a frame, a member, and a part is referred to as being “on” or “top (portion)” another portion, it may be directly on another element or may be on another element with the other element interposed therebetween.

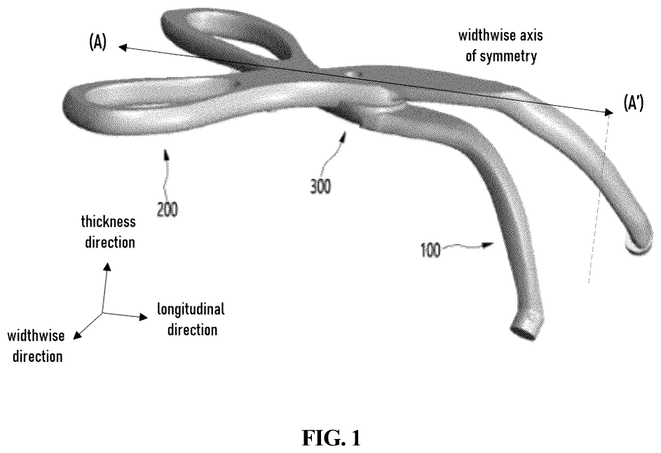

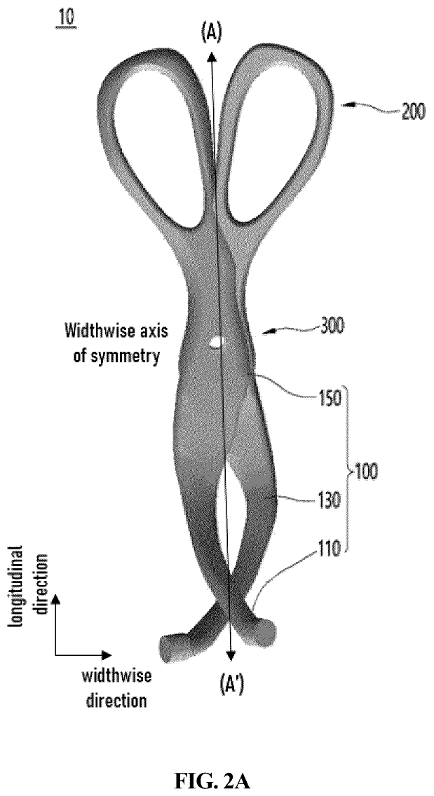

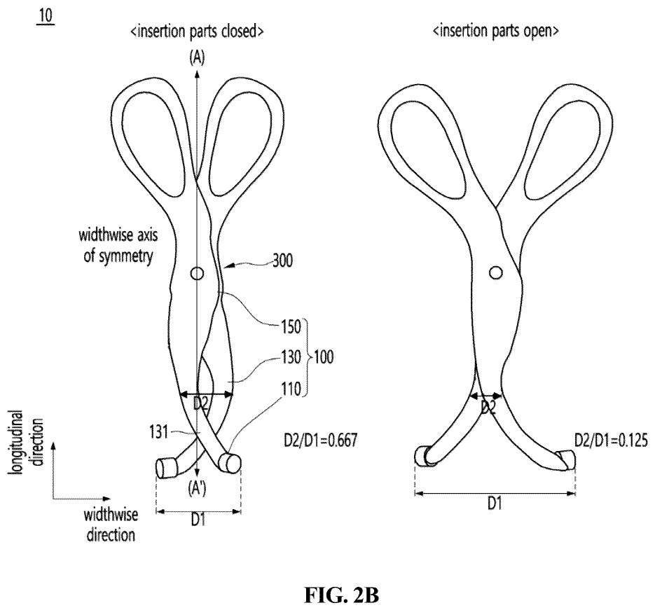

One embodiment of the present disclosure provides a pelvic floor stiffness measuring device. FIG. 1 is a schematic diagram of a pelvic floor stiffness measuring device including a pair of pressure members according to one embodiment of the present disclosure, FIG. 2A is a front schematic diagram of the pelvic floor stiffness measuring device in a closed state, based on a longitudinal direction of a pair of pressure members according to one embodiment of the present disclosure, and FIG. 2B is a front schematic diagram illustrating open and closed states of insertion parts of the pair of pressure members.

Referring to FIGS. 1, 2A, and 2B, the pelvic floor stiffness measuring device is configured to include, based on a longitudinal direction of a pair of pressure members, insertion parts 100 formed at each end of the pair of pressure members 10; grip parts 200 formed at each other end; and a hinge part 300 that does not intersect between the insertion part 100 and the grip part 200. The pair of pressure members may have a scissors shape in which the pair of insertion parts 100 are opened and closed by opening and closing the pair of grip parts 200 around the hinge part 300. According to the present disclosure, a user opens and closes the grip parts, so the pair of insertion parts inserted into vagina or anus around the hinge part are opened and closed while being in contact with the pelvic part. Accordingly, the principle is that the pair of insertion parts are pressurized on both wall surfaces of the pelvic floor tissue while being spaced apart from each other, and the movement distance of the tissue and the pressure at this time are measured to calculate the tissue stiffness. According to the present disclosure, there is an advantage in that the volume is small and the pelvic floor tone may be evaluated by accurately measuring the pressure element without finger palpation.

The pair of insertion parts 100 are inserted into the vagina or anus to directly contact and apply pressure to the pelvic floor, which is the target of the stiffness measurement, and each end forms a pelvic floor pressure area 110 based on the longitudinal direction of the pair of pressure members, and each other end is connected to the hinge part 300.

The insertion parts 100 are formed as a downward convex curved surface between the pelvic floor pressure area 110 and the hinge part 300 based on a widthwise axis of symmetry through which the pair of pressure members is open and closed, and are connected to an intermediate area 130 at which a point 131 where the pair of insertion parts intersect is formed in at least a part of the pair of insertion parts.

Meanwhile, as illustrated in FIGS. 1, 2A, and 2B, the widthwise axis of symmetry along which the pair of pressure members is open and closed means a straight axis connecting a center point A of the pair of grip parts 200 and a center point A′ of the pair of insertion parts 100, and is a central axis along which the pair of insertion parts are open and closed around the hinge part by the opening and closing of the pair of grip parts.

The present disclosure is different from a configuration of general scissors in which a grip part and a blade part (corresponding to an insertion part) contact and intersect at a hinge part (corresponding to the hinge part) and contact and do not intersect at an intermediate area of the blade part in that i) the pair of pressure members has a curved shape so as to contact but not intersect each other at the hinge part 300 connecting the insertion part 100 and the grip part 200 in the middle, and ii) the pair of insertion parts contact and intersect 131 at the intermediate area located between the hinge part and the insertion part (based on the widthwise axis of symmetry). Accordingly, when the pair of insertion parts is in a closed state (contracted state) as illustrated in FIG. 2A, the separation distance between the pair of pelvic floor pressure areas 110 is short, so the pair of insertion parts may be inserted into a narrow vagina or anus without any particular physical damage. Next, as illustrated in FIG. 2B, when the insertion part is open (in an expanded state) according to the opening of the grip part, the separation distance between the intermediate area 130 located in the opposite direction to the pelvic floor pressure area 110 based on the intersection point 131 tends to decrease and then increase, which may improve the problem of pressure dispersion by preventing the measuring device from contacting other internal body tissues other than the pelvic floor. Accordingly, the pelvic floor pressure areas 110 formed at each end of a pair of pressure members 10 may efficiently contact and pressurize only both wall surfaces of the pelvic floor tissue.

In addition, iii) since the intermediate area is provided with a downward convex curved surface (based on the widthwise axis of symmetry), in addition to the effect of simply preventing damage to the body by forming the insertion part surface as the curved surface, the above-described effect may be further improved. On the other hand, when it is provided with an upward convex curved surface or a flat surface, a variation range in the separation distance between the intermediate areas may increase when the insertion part is open and closed, so the effect of preventing the contact and pressure from being distributed to other internal body tissues other than the pelvic floor may be relatively reduced.

In addition, the pair of insertion parts 100 may be characterized by satisfying the following Relational Expression 1.

$$0 \leq D 2 / D 1 < 1 \qquad [ Relational Expression 1 ]$$

In Relational Expression 1, D1 and D2 each are the separation distance D1 between the pair of pelvic floor pressure areas 110 and the separation distance D2 between the pair of intermediate areas 130 based on the widthwise axis of symmetry of the pair of pressure members 10 when the pair of insertion parts 100 is open and closed by the opening and closing of the pair of grip parts 200, respectively.

In the Relational Expression 1, 0<D2/D1<1, 0.1<D2/D1<0.9, 0.1<D2/D1<0.8 or 0.1<D2/D1<0.7 may be satisfied. In the present disclosure, the Relational Expression 1 may be satisfied in a state where the grip part and the insertion part are closed (maximally contracted). In this case, 0.5<D2′/D1′<0.75 or 0.6<D2′/D1′<0.7 may be satisfied (Relational Expression 1-1). Accordingly, when inserting the measuring device into the vagina or the anus, it is possible to insert the measuring device into a narrow vagina or anus without any particular physical damage (see <closed state of insertion part> of FIG. 2B). In addition, in the present disclosure, the Relational Expression 1 may be satisfied in the state where the grip part and the insertion part are open (from a completely contracted state to a slowly expanded state) after the insertion part is inserted into the vagina or the anus. In this case, 0<D2/D1<0.5, 0<D2/D1<0.3 or 0.1<D2/D1<0.2 may be satisfied (Relational Expression 1-2). By reducing the variation range of the separation distance D2 in the intermediate area when opening the insertion part after the insertion and relatively increasing the variation range of the separation distance D1 in the pressure area, the problem of contact and pressure being distributed to other internal body tissues other than the pelvic floor when opening the insertion part in a narrow and winding a vagina or anal passage may be improved (see <open state of insertion part> in FIG. 2B).

Here, the separation distance D2 of the pair of intermediate areas 130 may be the average separation distance of at least a portion of the pair of intermediate areas, and may be the average separation distance of all areas other than each pelvic floor pressure area 110 and the lower safety region 150 in the pair of insertion parts based on the longitudinal direction of the pair of pressure members 10, and preferably, may be the separation distance of the area with the longest or shortest separation distance among the pair of intermediate areas when the pair of insertion parts is closed.

In addition, the pair of insertion parts 100 may further include a safety area 150 in which each end is connected to the hinge part 300. Reflecting this, when the pair of insertion parts 100 changes from a closed state to an open state by opening and closing the pair of grip parts 200, the pair of pelvic floor pressure areas 110 may indicate a gradient in which the separation distance D1 increases, the pair of intermediate areas 130 may indicate a gradient in which the separation distance D2 decreases and then increases. In addition, conversely, when the pair of insertion parts 100 changes from the open state to the closed state by opening and closing the pair of grip parts 200, the pair of pelvic floor pressure areas 110 may indicate a gradient in which the separation distance D1 decreases, the pair of intermediate areas 130 may indicate the gradient in which the separation distance D2 increases and then decreases. Therefore, the above-described effect in the present invention may be further improved.

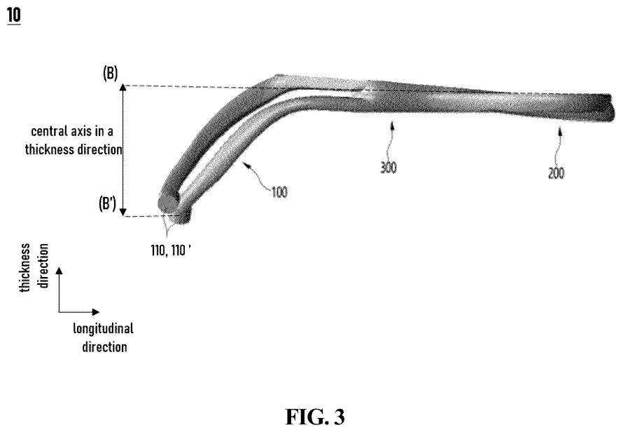

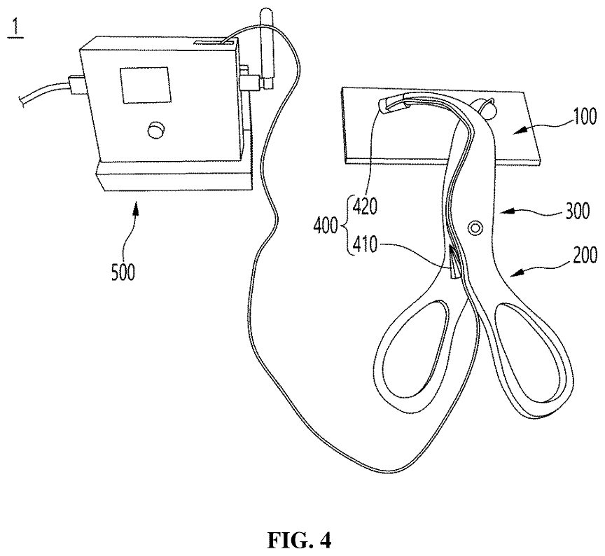

FIG. 3 is a schematic diagram of the pelvic floor stiffness measuring device in the closed state, based on a thickness direction of the pair of pressure members according to one embodiment of the present disclosure.

Referring to FIG. 3, the pair of insertion parts may be provided such that, based on the central axis in the thickness direction of the pair of pressure members 10, each pelvic floor pressure area 110 may be i) located under the hinge part 300, and ii) provided in an upward convex shape.

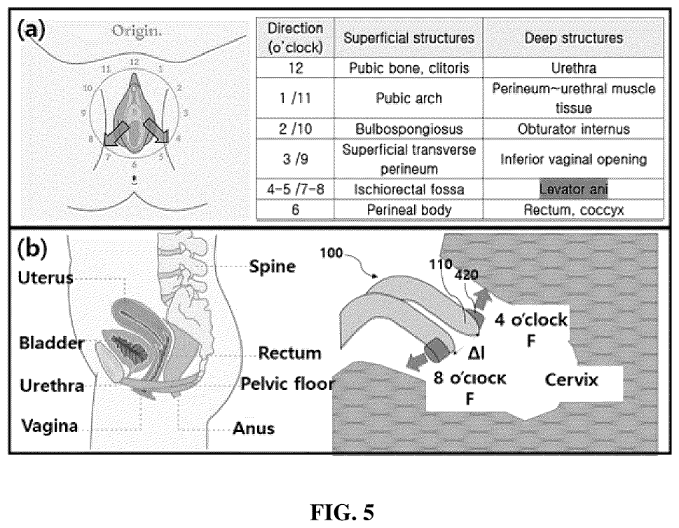

Accordingly, since each pelvic floor pressure area 110 is i) located under the hinge part 300, the pelvic floor pressure area 110 of the pair of insertion parts is easily inserted downward when inserted into the vagina or anus, so, as in FIG. 5, when the front of the introitus or the anus opening approximate a uniform circle, the pressure area 110 of the insertion parts may effectively reach the levator ani, which is a deep structure of the lower pelvic floor, located at 4 to 5 o'clock and 7 to 8 o'clock. In addition, ii) since the pelvic floor pressure area 110 is provided in an upward convex shape, when generally approximated as a lying cylindrical shape, there is a tendency for the separation distance between two wall surfaces of the pelvic floor to narrow as one goes down to the lower pelvic floor in the thickness direction from the vaginal or anal passage, the present disclosure may prevent contact with and pressure from being distributed to other internal body tissues other than the pelvic floor. On the other hand, when the pelvic floor pressure area is provided in a downward convex shape, it may not be easy to open and close the insertion part within the pelvic cavity, and in addition, areas of other insertion part 100 such as the intermediate area 130 and the safety area 150 may come into contact with the deep structures of the lower pelvic floor, which is not preferable.

Meanwhile, as illustrated in FIG. 3, the central axis in the thickness direction of the pair of pressure members 10 means a straight axis connecting a center point B of the pair of grip parts 200 and a center point B′ of the hinge part 300 based on the thickness direction.

The pair of grip parts 200 may have each end located on the opposite side to the insertion part and formed in a loop shape, and may have a first finger hole into which a thumb is inserted and a second finger hole into which a finger other than the thumb is inserted. The first and second finger holes may have a circular, oval or egg shape. In addition, each other end of the grip part 200 may be formed to extend each end and be connected to the pair of hinge parts.

The above-described pair of hinge parts 300 is located between the insertion part 100 and the grip part 200, and the pair of pressure members 10 are formed so as to contact but not intersect at the hinge part, and also, first and second protrusions (not illustrated) may be formed that protrude from the surface in the thickness direction of the pair of pressure members 10. When the first and second protrusions are formed with through holes in an overlapping state, an axis may be passed through the holes to fix both ends so that the first and second protrusions may rotate. Accordingly, the pair of insertion parts 100 is configured to be open and closed by opening and closing the pair of grip parts 200 around the hinge part 300 in the pair of pressure members 10.

The pair of pressure members 10 may further include a sensor unit 400 that includes a motion 410 and a pressure sensor 420 for calculating the stiffness of the pelvic floor. According to the present disclosure, when the pair of insertion parts 100 is open and closed by opening and closing the pair of grip parts 200, the movement sensor 410 may measure the movement distance of the pair of pelvic floor pressure areas 110, and the pressure sensor 420 may measure pressurized force on the pelvic floor.

The pressure sensor 420 may be installed in the pelvic floor pressure area 110 of the pair of insertion parts 100 and/or at least some area of the pair of grip parts 200, and preferably, may be installed in the pelvic floor pressure area 110 of the pair of insertion parts 100 or at least a portion of the pair of grip parts 200.

For example, after the pelvic floor pressure area 110 of the insertion part is brought into contact with the pelvic floor deep structure, the insertion part is expanded in the width direction to move the pelvic floor pressure area. The pressure applied to the pelvic floor is measured through the pressure sensor installed in the pelvic floor pressure area, and the pressure information is output. The movement distance of the pelvic floor pressure area and the movement distance of the tissue of the pelvic floor deep structure are measured through the movement sensor, and the movement distance information is output. The stiffness of the pelvic floor deep structure may be calculated from the pressure information and the movement distance information.

The movement sensor 410 may output the movement distance information by calculating the change in length of each (insertion part) pelvic floor pressure area 110 when a center angle between the pair of insertion parts 100 based on the hinge part 300 changes as the pair of pressure members 10 are open and closed in the width direction. In this case, the change in length may mean the change in length of an arc or string according to the change in the central angle, but the present disclosure is not limited thereto.

The pelvic floor stiffness measuring device according to one embodiment may further include a control unit 500. Accordingly, the pressure sensor 420 is disposed in the pelvic floor pressure area 110 of the insertion part 100, and a movement distance measuring module (not illustrated) and a pressure measuring module (not illustrated) are built into the control unit 500, so that after the insertion part 100 is inserted into the vagina or anus, the grip part 200 is open and closed to apply pressure to the pelvic floor, and then, the applied pressure and movement distance are measured to output the pressure information and the movement distance information. Next, a stiffness calculation module (not illustrated) is built into the control unit 500, so that the pelvic floor tissue stiffness may be calculated through the pressure information and the movement distance information.

Hereinafter, the operating state of the pelvic floor stiffness measuring device according to the present disclosure will be described in detail with reference to the drawings.

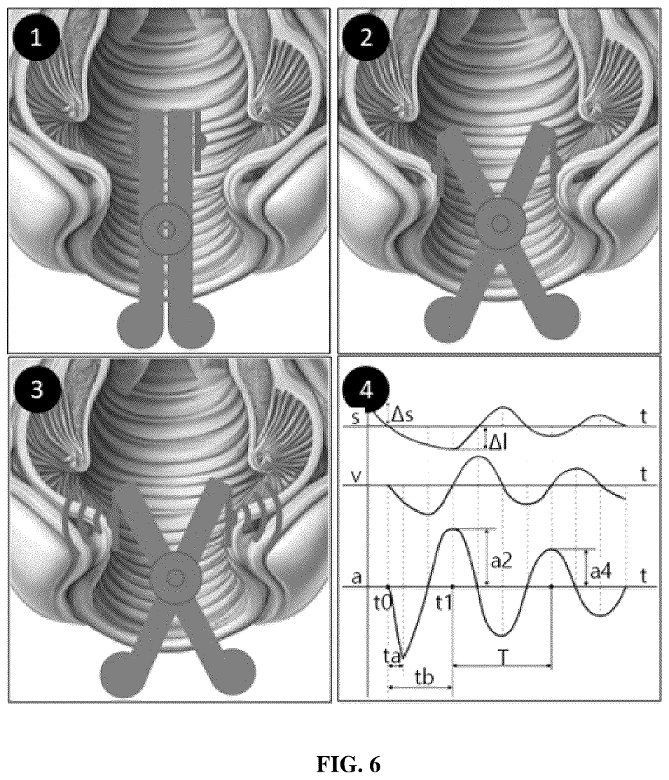

FIGS. 5 and 6 are reference diagrams for describing an operating state of the pelvic floor stiffness measuring device according to the present disclosure.

As illustrated in FIG. 5, in order to reach the pressure area 110 of the insertion part to the levator ani, which is the deep structure of the lower pelvic floor located in 4 to 5 o'clock and 7 to 8 o'clock directions when the front of the introitus or the anus opening approximates a uniform circle, the pair of pressure members 10 of the pelvic floor stiffness measuring device are adjusted to be closed so that one end portion (pelvic floor pressure area) of the insertion part 100 is contracted in the width direction, and as a result, the pair of pressure members 10 contacts the introitus and the hinge part 300 is adjusted to be located at the center of the front of the introitus or the anus opening. In this case, the insertion part is slowly inserted into the introitus while keeping the grip part 200 closed, but carefully inserted by minimizing the distance between both end portions of the pair of insertion parts contacting the introitus (see \textcircled{1} of FIG. 6).

The pair of pressure members 10 are slowly open (expanded) in a widthwise opening (expanded) distance of about 6 to 8 cm to find a point where a valid pressure change (the point at which external force begins to act upon the tissue) appears on the pressure sensor 420, and the distance between the two pelvic floor pressure areas 110 is output (marked) (see \textcircled{2} of FIG. 6).

While applying a certain downward pressure to fix the insertion part, the external force applied to the range where the pelvic floor tissue no longer moves is measured while opening (expanding) the pressure area 110 of the insertion part (see \textcircled{3} of FIG. 6).

Then, the output external force is divided by the movement distance to calculate the tissue stiffness (see \textcircled{4} of FIG. 6).

The pelvic floor tissue stiffness measuring device of the present disclosure may be used as equipment that pelvic floor health experts (doctors, physical therapists, nurses, etc.) may easily measure in clinical practice. For example, objective information on the pelvic floor tissue tone may be screened in private clinics that are not equipped with imaging equipment. The clinical significance of the pelvic floor tissue tone is related to the function of pelvic floor muscles, and pelvic floor muscle dysfunction may be associated with various symptoms such as urinary incontinence, sexual dysfunction, pelvic floor pain, nonbacterial prostatitis, frequent urination, residual urine, chronic constipation, and fecal incontinence. In particular, symptoms such as frequent urination, residual urination, dyspareunia, and pelvic floor pain are considered to be associated with high pelvic floor tissue tone. On the other hand, urinary incontinence and pelvic organ prolapse (urethral prolapse, cystocele, uterine prolapse, rectal prolapse, etc.) are associated with the low pelvic floor tissue tone. Accordingly, according to the present disclosure, the pelvic floor tissue stiffness measuring device may be usefully utilized to obtain the measurement values and standard deviations of normal people, obtain the pelvic floor tissue tone information for each disease based on the obtained measurement values and standard deviations, and then determine therapeutic methods for normalizing the tone information.

### DESCRIPTION OF THE SIGN

1: pelvic floor stiffness measuring device
10: pressure members
100: insertion parts
110: pelvic floor pressure area
130: intermediate area
131: a point where the pair of insertion parts intersect
150: safety area
200: grip parts
300: hinge part
400: sensor unit
410: movement sensor
420: pressure sensor
500: control unit

1. A pelvic floor stiffness measuring device, the device comprising:
a pair of longitudinal pressure members that includes a pair of insertion parts respectively disposed at first portions of the pair of longitudinal pressure members, and a pair of grip parts respectively disposed at second portions of the pair of longitudinal pressure members, the second portions being opposite the first portions with respect to a hinge,
wherein the longitudinal pressure members contact, but do not interest, each other at the hinge in a width direction thereof, and the insertion parts are configured to be opened or closed by opening and closing the grip parts around the hinge,
wherein each of the insertion parts includes a pelvic floor pressure area disposed at an end thereof,
wherein each of the insertion parts has a downward convex curved surface based on a widthwise axis of symmetry, and
wherein each of the insertion parts comprises an intermediate area, and at least a portion of the intermediate area comprises a point where the insertion parts intersect.

2. The pelvic floor stiffness measuring device of claim 1, wherein
the pair of insertion parts satisfies the following Relational Expression 1:
$$0 \leq D 2 / D 1 < 1 ,$$
wherein D1 and D2 each are a separation distance between the pelvic floor pressure areas and a separation distance between the intermediate areas based on the widthwise axis of symmetry of the pair of longitudinal pressure members when the insertion parts are opened or closed by the opening and closing of the grip parts, respectively.

3. The pelvic floor stiffness measuring device of claim 1, wherein
the pair of insertion parts further respectively includes a safety area connected to the hinge, and
when the pair of insertion parts changes from a closed state to an open state by opening and closing the pair of grip parts, a separation distance between the pelvic floor pressure areas increases, and a separation distance between the intermediate areas decreases and then increases.

4. The pelvic floor stiffness measuring device of claim 1, wherein
the insertion parts are configured such that, based on a central axis in a thickness direction of the pair of longitudinal pressure members, each of the pelvic floor pressure areas is located under the hinge and has an upward convex shape.

5. The pelvic floor stiffness measuring device of claim 1, wherein each of the longitudinal pressure members further includes a sensor unit that includes a movement sensor configured to measure a movement distance of the pelvic floor pressure areas and a pressure sensor configured to measure pressurized force on the pelvic floor, when the insertion parts are opened or closed by opening and closing the grip parts.

6. The pelvic floor stiffness measuring device of claim 5, wherein
the pressure sensor is installed in the pelvic floor pressure area of each of the pair of insertion parts and/or at least an area of the pair of grip parts.

7. (canceled)
